SEQUENCE LISTING (3 sequences)

SEQ ID NO: 1 - RNA, 23 nt, synthetic construct
  source 1..23: mol_type = other RNA
  modified_base 1: mod_base = OTHER; note = phosphorothiolated
  modified_base 2: mod_base = OTHER; note = phosphorothiolated
  modified_base 3: mod_base = OTHER; note = phosphorothiolated
  modified_base 20: mod_base = OTHER; note = phosphorothiolated
  modified_base 21: mod_base = OTHER; note = phosphorothiolated
  modified_base 22: mod_base = OTHER; note = phosphorothiolated
  modified_base 23: mod_base = OTHER; note = Cy5
gtccagttttcccaggaatccct

SEQ ID NO: 2 - RNA, 22 nt, synthetic construct
  source 1..22: mol_type = other RNA
  modified_base 1: mod_base = OTHER; note = phosphorothiolated
  modified_base 2: mod_base = OTHER; note = phosphorothiolated
  modified_base 3: mod_base = OTHER; note = phosphorothiolated
  modified_base 19: mod_base = OTHER; note = phosphorothiolated
  modified_base 20: mod_base = OTHER; note = phosphorothiolated
  modified_base 21: mod_base = OTHER; note = phosphorothiolated
  modified_base 22: mod_base = OTHER; note = ATTO488
caatcagcaagtatactgccct

SEQ ID NO: 3 - RNA, 22 nt, synthetic construct
  source 1..22: mol_type = other RNA
  modified_base 1: mod_base = OTHER; note = phosphorothiolated
  modified_base 2: mod_base = OTHER; note = phosphorothiolated
  modified_base 19: mod_base = OTHER; note = phosphorothiolated
  modified_base 20: mod_base = OTHER; note = phosphorothiolated
  modified_base 21: mod_base = OTHER; note = phosphorothiolated
  modified_base 22: mod_base = OTHER; note = TYE563
tcaacatcagtctgataagcta

(19) United States
(12) Patent Application Publication
(10) Pub. No.: US 2025/0367133 A1
(43) Pub. Date: Dec. 4, 2025

(54) POLYMER NANOPARTICLE COMPOSITIONS FOR NON-VIRAL GENE DELIVERY

(71) Applicant: BATTELLE MEMORIAL INSTITUTE, Columbus, OH (US)

(72) Inventors: Ashlee J. Colbert, Blacklick, OH (US); Emma K. Schmitz, Columbus, OH (US); Kenneth R. Sims, JR., Delaware, OH (US); Andrea D. McCue, Columbus, OH (US); Allison Secard, Columbus, OH (US)

(21) Appl. No.: 19/225,707

(22) Filed: Jun. 2, 2025

Related U.S. Application Data
(60) Provisional application No. 63/654,480, filed on May 31, 2024.
(60) Provisional application No. 63/776,648, filed on Mar. 24, 2025.
(60) Provisional application No. 63/779,213, filed on Mar. 27, 2025.

(51) Int. Cl. A61K 9/51; C08F 293/00; C12N 15/113; C12N 15/88

(52) U.S. Cl. CPC A61K 9/5138; A61K 9/5146; C08F 293/00; C12N 15/113; C12N 15/88; C12N 2310/141

(57) ABSTRACT

The disclosure relates to block copolymer nanoparticles for therapeutic delivery of nucleotides, and methods therefor. More particularly, the invention relates to polymer nanoparticles, such as reversible addition-fragmentation chain transfer (RAFT) polymer compositions, for delivering miRNAs.

### RELATED APPLICATIONS

This application claims the benefit of U.S. Provisional Application No. 63/779,213, filed Mar. 27, 2025, U.S. Provisional Application No. 63/776,648, filed Mar. 24, 2025, and U.S. Provisional Application No. 63/654,480, filed May 31, 2024, the entire disclosure of each of which is incorporated herein by reference.

### INCORPORATION BY REFERENCE OF MATERIAL SUBMITTED ELECTRONICALLY

Incorporated by reference in its entirety is a computer-readable nucleotide sequence listing submitted concurrently herewith and identified as follows: 22 kilobytes xml file named “422713.xml”, created on May 29, 2025.

### BACKGROUND

Genetic medicines (including gene therapy, gene silencing, splicing regulators, and nuclease based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Adeno-associated viruses (AAVs) are the most widely used tool for genetic medicine delivery, but AAVs are unable to deliver multiple payloads efficiently (such as co-delivery of small non-coding micro RNAs (miRNAs)) and they sometimes trigger unwanted immune responses, including the generation of anti-AAV antibodies, a cell mediated response. Some of the immune responses caused by AAV in patients are potentially fatal immune responses.

miRNA based therapeutics have an exceptional potential to treat a number of genetic diseases due to their ability to regulate gene expression post-transcriptionally. In cancer, miRNAs that regulate oncogenes or tumor suppressor genes can be modulated to inhibit tumor growth or induce apoptosis in cancer cells. However, the main challenge is off-target effects and efficient delivery of the miRNAs which can modulate multiple genes. This requirement becomes particularly limiting for in vivo applications because ensuring co-delivery of multiple miRNAs to the same targeted location is currently not feasible.

Lung cancer therapies including surgery, radiation therapy, and chemotherapy have severe side effects and limited efficacy, particularly in advanced stages of the disease. Thus, there is a need for effective non-viral delivery systems, including gene delivery systems for treating lung cancer. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

### SUMMARY

In certain aspects, a block copolymer comprises

a first block comprising a homopolymer of monomer units represented by formula I:

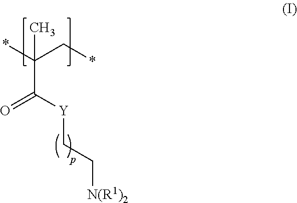

or a salt thereof, wherein:
p is 0 or an integer selected from 1-3,

each R¹ is individually selected from H or alkyl;

Y is O or NH; and

wherein the * represent the connecting points to the rest of the block copolymer; and

a second block comprising either:
(i) a homopolymer comprising monomer units represented by formula II:

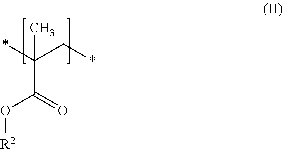

or a salt thereof, wherein:
R² is alkyl optionally substituted by hydroxy; and

wherein each * represents a connecting point to the rest of the block copolymer; or

(ii) a copolymer comprising at least two different monomer units, each represented by formula III:

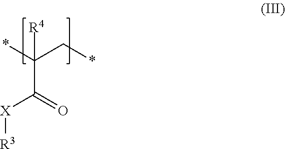

or a salt thereof, wherein:
X is O or NR⁵;
R³ is alkyl optionally substituted by amino;
R⁴ is H or alkyl; and
R⁵ is alkyl;
wherein each * represents a connecting point to the rest of the block copolymer.

In certain aspects, a block copolymer comprises a first block and a second block, wherein: the first block comprises a homopolymer of poly (2-dimethylaminoethyl methacrylate (p(DMAEMA)) or poly 2-aminoethyl methacrylamide (p(AEMA)); and

the second block comprises either:
(i) a homopolymer of butylmethacrylate (BMA) or a homopolymer of hydroxyethylmethacrylate (HEMA); or
(ii) a copolymer comprising at least two monomer units selected from DMAEMA, BMA, and diethylacrylamide (DEAAM).

In certain aspects, a polymer nanoparticle comprises a block copolymer according to the present disclosure.

In certain aspects, a composition comprises a polymer nanoparticle according to the present disclosure and a nucleic acid complexed to the polymer nanoparticle.

In certain embodiments, the nucleic acid is a miRNA.

In certain aspects, a composition comprises a polymer nanoparticle according to the present disclosure and at least two different nucleic acids (e.g., miRNAs) complexed to the polymer nanoparticle.

In certain aspects, a method of treating a disease comprising administering a therapeutically effective amount of a composition according to the present disclosure to a patient in need thereof. In certain aspects, a method of treating a lung disease is provided.

In certain aspects, a method of transfecting a cell comprising contacting the cell with an effective amount of a composition according to the present disclosure.

Additional embodiments, features, and advantages of the disclosure will be apparent from the following detailed description and through practice of the disclosure.

### BRIEF DESCRIPTION OF THE DRAWINGS

FIG. 1 shows Dynamic Light Scattering (DLS) analysis of the polymer nanoparticles in multiple synthesis points including prior to lyophilization, and post lyophilization

FIGS. 2A-2B show flow cytometer analysis of the transfection efficiency (TE%) in A549 lung cancer cells following 48 hrs. post transfection with miR34, anti-miR-21, and miR145. TE% was calculated for cells dosed with freshly prepared and loaded PNPs, as in FIG. 2A. PNPs lyophilized with miRNA are shown in FIG. 2B.

FIGS. 3A-3C display the qPCR analysis of PNP carriers showing PNP carriers effectively deliver miRNA and regulate endogenous expression accordingly compared to miRNA scramble controls. Specifically, FIG. 3A shows the relative fold change of miR145 and scramble (Scr) in various carriers, FIG. 3B, the relative fold change for miR34a versus scramble (Scr), and in FIG. 3C, the relative fold change for miR21 versus scramble control (Scr).

FIG. 4 shows percent wound closure at 36 hours of the PNPs compared to Lipofectamine and miRNA control.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or

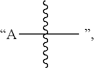

where each of“-*”, “-**”, and

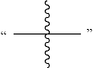

represents a bond to A and the point of covalent bond attachment to B. Alternatively, in some embodiments, the portion of A-B defined by the group or chemical structure B can be represented by “*-B”, “**-B”, or

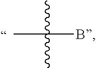

where each of “*”, “-**”, and

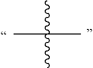

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### CHEMICAL DEFINITIONS

The term “alkyl” refers to a straight-or branched-chain monovalent hydrocarbon group. The term “alkylene” refers to a straight-or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—-)₃), iso-propylene ((—C(H)(CH₃)CH₂—)), n-butylene ((—CH₂—)₄), and the like. It will be appreciated that an alkyl or alkylene group can be unsubstituted or substituted as described herein. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including hydroxy groups such as 1-hydroxyethanol. An alkyl or alkylene group can be substituted with any of the substituents in the various embodiments described herein, including one or more of such substituents.

The terms “amine” and “amino” are art-recognized and refer to both unsubstituted and substituted amines and salts thereof, e.g., a moiety that can be represented by

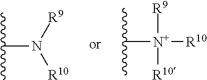

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ₋(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. ⊖,”wherein “W⊖” is an inorganic counter ion (e.g., an inorganic anion) or an organic counter ion (e.g., an organic anion). In certain embodiments, W⊖ is an anion that is complexed with a cation of a compound of the disclosure to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound (e.g., a salt or a pharmaceutically acceptable salt) or can substituted to a salt for further manufacture, formulation, or administration reasons. As illustrated herein, certain compounds include a “W⊖,” wherein “W⊖” is an inorganic counter ion (e.g., an inorganic anion) or an organic counter ion (e.g., an organic anion). In certain embodiments, W⊖ is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

As used herein, “nucleic acid” refers to a polymeric form of nucleotides of any length, either ribonucleotides or deoxyribonucleotides. This term refers only to the primary structure of the molecule. Thus, this term includes double and single stranded DNA, triplex DNA, as well as double and single stranded RNA (e.g., microRNA or miRNA), linear RNA, circular RNA, and other RNA formats. It also includes modified forms, for example, by methylation and/or by capping, and unmodified forms of the polynucleotide. The term is also meant to include molecules that include non-naturally occurring or synthetic nucleotides as well as nucleotide analogs.

### REPRESENTATIVE EMBODIMENTS

This disclosure describes compositions of cationic polymers (e.g., diblock copolymers) which self-assemble in aqueous conditions to form polymer nanoparticles (PNPs). In illustrative embodiments, the PNPs can complex nucleic acids and deliver the complexed nucleic acids to a desired location. In illustrative embodiments, the diameter of the PNPs is similar to the diameter of the PNPs when complexed to nucleic acids.

MicroRNAs (miRNAs) are emerging as a novel approach in lung cancer therapy due to their ability to regulate gene expression, however current techniques are unable to accurately target and deliver them to the right cells. To function properly, these small, non-coding RNA molecules need to inhibit the translation of specific messenger RNAs (mRNAs) in cancer cells, thereby controlling the production of proteins that are involved in cell growth, differentiation, and death. In the context of lung cancer, certain miRNAs have been found to be dysregulated, leading to uncontrolled cell proliferation and tumor growth. By introducing or inhibiting these specific miRNAs, researchers can potentially alter the behavior of cancer cells. For instance, miRNAs that act as tumor suppressors can be introduced to inhibit tumor growth, while those that promote cancer can be inhibited.

Cationic polymeric nanoparticles (PNPs) offer advantages in cargo capacity, and delivery efficiency with reduced toxicity when compared to other delivery vehicles such as adeno- associated virus (AAV) vectors. Additionally, the capacity of these PNPs allows for delivery of multiple miRNAs and potentially combinatorial therapies. miRNA mimics were successfully delivered for RNA replacement therapy alongside miRNA inhibitors loaded in a single nanoparticle in vitro. This delivery to lung cancer cells has been confirmed via qPCR and flow cytometry. Furthermore, the efficacy of these loaded nanoparticles post-lyophilization has been evaluated with confirmed delivery in vitro. Lyophilization increases shelf-life and stability of the loaded nanoparticles and creates a product that is easy to transport and store.

Targeted PNPs as a delivery vehicle for multiple dysregulated miRNAs in lung cancer may result in a therapy that increases survival rates and improves the quality of life for patients with lung cancer. miRNA therapeutics may also provide a platform to treat other cancers and diseases with miRNA biomarkers, thereby creating a foundation for increasing survival rates and improving quality of life for all.

Nanoparticles were designed for delivery to the lungs. These nanoparticles were synthesized and tested in vitro for delivery of a cocktail containing two miRNA mimics and one miRNA inhibitor. Furthermore, relative fold change for the expression of micro RNA miR-145 and miR-34a was found to be increased more than 100-fold for some nanoparticles while the fold change was reduced for the miRNA inhibitor, miR-21, when compared to controls (qPCR). In addition, lung cells were transfected with the miRNA cocktail and monitored over time to determine the wound closure after 36 hours. It was seen that the miRNA cocktail improved wound closure in the cultured lung cells.

In certain embodiments, a block copolymer comprises a first block and a second block. The first block may comprise a homopolymer of poly (2-dimethylaminoethyl methacrylate) (pDMAEMA) or a homopolymer of p(AEMA). The second block may be a homopolymer or a copolymer of two or more unique monomer units. In some embodiments, the second block comprises a homopolymer of p(BMA) or a homopolymer of p(HEMA). In some embodiments, the second block comprises a copolymer of BMA and DEAAM.

In certain embodiments, a block copolymer includes a first block having a monomer unit represented by formula I:

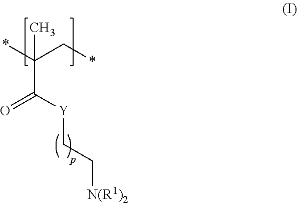

or a salt thereof, wherein:
p is 0 or an integer selected from 1-3,
each R¹ is individually selected from H or alkyl;
Y is O or NH; and

wherein each * represents a connecting point to the rest of the block copolymer.

In certain embodiments, p is 0. In certain embodiments, p is 1, 2 or 3, and is preferably 1.

In certain embodiments, each R¹ is individually selected from H or alkyl (e.g., C₁-C₃ alkyl). In certain embodiments, each R¹ is alkyl (e.g., C₁-C₃ alkyl) and each alkyl (e.g., C₁-C₃ alkyl) may be the same. In certain preferred embodiments, each R¹ is H or methyl. In certain embodiments, the first block comprises a plurality monomers of formula I such as

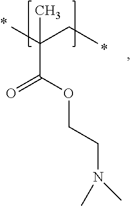

or a salt thereof, the corresponding polymer of which may be called poly(2-(dimethylamino)ethyl methacrylate) p(DMAEMA). In certain embodiments, the first block comprises a plurality monomers of formula I such as

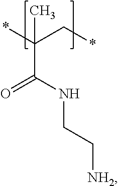

or a salt thereof, the corresponding polymer of which may be called poly(aminoethylmethacrylamide) p(AEMA).

In certain embodiments, the second block comprises a homopolymer comprising monomer units represented by formula II:

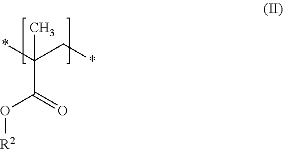

or a salt thereof, wherein:

R² is alkyl optionally substituted by hydroxy; and

wherein each * represents a connecting point to the rest of the block copolymer.

For example, the monomer unit of formula II may be

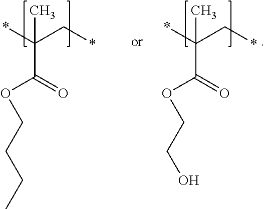

In certain embodiments, the second block comprises a homopolymer of poly(butylmethacrylate) p(BMA) or a homopolymer of poly(2-hydroxyethylmethacrylate) p(HEMA).

In certain embodiments, the second block comprises a copolymer comprising at least two different monomer units, each represented by formula III:

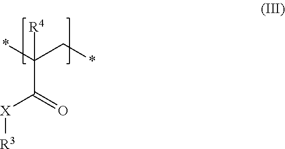

or a salt thereof, wherein:

X is O or NR⁵;

R³ is alkyl optionally substituted by amino;

R⁴ is H or alkyl; and

R⁵ is alkyl;

wherein each * represents a connecting point to the rest of the block copolymer.

In certain embodiments, R³ is alkyl, such as C₁-C₆ alkyl (e.g., ethyl or butyl), and is preferably ethyl or butyl. For example, the monomer represented by formula III may be:

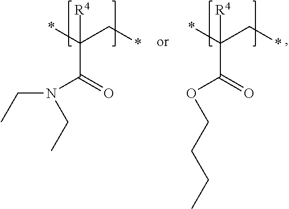

or a salt thereof. In certain embodiments, R³ is unsubstituted.

In certain embodiments, R⁴ is alkyl such as C₁-C₆ alkyl, and is preferably methyl. For example, the monomer unit represented by formula III may be:

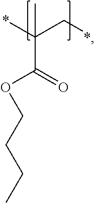

or a salt thereof, which may be referred to as butylmethacrylate (BMA), when present in the block copolymer. In certain embodiments, R⁴ is H. For example, the monomer unit represented by formula III may be

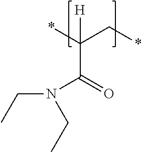

or a salt thereof, which may be referred to as N,N-Diethylacrylamide (DEAAM), when present in the block copolymer.

In certain embodiments, R⁵ is C₁-C₆ alkyl (e.g., methyl or ethyl, and preferably is ethyl).

In certain embodiments, the first block has a molecular weight of about 2,000 Da to about 40,000 Da. For example, the first block may be about 2,000 Da to about 12,000 Da, or about 25,000 Da to about 40,000 Da.

In certain embodiments, the first block has a degree of polymerization of about 20 to about 250. For example, the first block may have a degree of polymerization of about 150 to about 250, or about 20 to about 90.

In certain embodiments, the second block has a degree of polymerization of about 2 to about 355. For example, the second block may have a degree of polymerization of about 1 to about 10, about 30 to about 70, about 70 to about 80, or about 330 to about 360.

In certain embodiments, the mol percent (mol %) of each of the compounds of formula III in the second block may be present at a particular ratio. For example, in certain embodiments the ratio of molecular weights of each of the monomers of formula III in the second block is about 20:80 to about 80:20, about 30:70 to about 70:30, or about 40:60 to about 60:40. In certain embodiments, the ratio of molecular weights of each monomer of formula III in the second block is about 60:40 or about 55:45.

In certain embodiments, the second block copolymer is a copolymer of at least two monomers, wherein the molar percent of the first monomer is about 30% to about 70% and the molar percent of the second monomer is about 70% to about 30%. In certain embodiments, the second block copolymer is a copolymer of at least two monomers, wherein the molar percent of the first monomer is about 50% to about 60% and the molar percent of the second monomer is about 50% to about 40%.

In some embodiments, block copolymers as prepared herein can be described by the following structure:

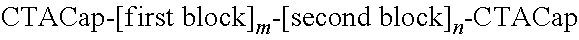

where each CTACap is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

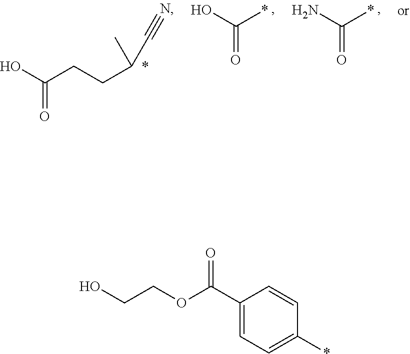

or a salt thereof, wherein * represents a point of covalent attachment to the first block. In certain preferred embodiments, the cap is

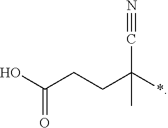

In some embodiments, the second capping unit is of formula

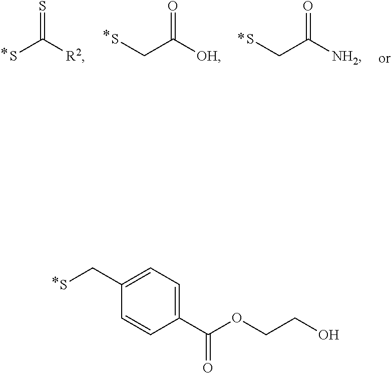

or a salt thereof, wherein * represents a point of covalent attachment to the second block, and R² is —SC₂-C₁₂ alkyl or C₆H₅, and is preferably

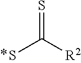

such as

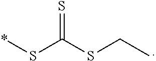 [SMILES: *SC(=S)SCC]

In some embodiments, a polymer nanoparticle comprises a block copolymer according to the present disclosure. In some embodiments, the block copolymer self-assembles into the nanoparticle.

In some embodiments, the diameter (e.g., a hydrodynamic diameter as measured by DLS) of the nanoparticle is about 200 nm to about 1000 nm, about 800 nm to about 1000 nm or about 200 nm to about 800 nm.

In some embodiments, the diameter (e.g., a hydrodynamic diameter as measured by DLS) of the nanoparticle changes following lyophilization. For example, the nanoparticle diameter can decrease in diameter following lyophilization. The polymer nanoparticle diameter can also be maintained following lyophilization.

In some embodiments, the diameter of the nanoparticle following lyophilization decreases to about 20 nm to about 1250 nm, about 20 nm to about 250 nm, about 20 to about 500 nm, or about 20 nm to about 850 nm.

In some embodiments, the diameter (e.g., a hydrodynamic diameter as measured by DLS) of the nanoparticle is about 200 nm to about 500 nm, or about 350 nm to about 750 nm, or about 850 nm to about 1250 nm.

In certain embodiments, the diameter of the nanoparticle increases following lyophilization. In certain embodiments, the diameter of the nanoparticle does not change following lyophilization.

In certain embodiments, the block copolymer has a measured molecular weight by NMR of about 2 kDa to about 60 kDa. In certain embodiments, the block copolymer has a measured molecular weight by NMR of about 35 kDa to about 45 kDa.

In some embodiments, the nanoparticle diameter is maintained following payload (e.g., nucleic acid) loading. For example, the nanoparticle has a diameter (as measured by DLS) prior to payload loading and a diameter after payload loading that is the same as the diameter prior to payload loading.

In some embodiments, a composition comprises a polymer nanoparticle as described herein and a nucleic acid (sometimes called a payload) complexed to the polymer nanoparticle. For example, the nucleic acid may be complexed to the nanoparticle through electrostatic interactions. In certain preferred embodiments, the polymer nanoparticle of the composition serves as a transfection agent to deliver a nucleic acid to a cell.

In certain embodiments, the nucleic acid is an RNA (e.g., an mRNA, a microRNA, antisense oligonucleotides, or an siRNA) or a DNA (e.g., an ssDNA, a dsDNA, or a complimentary coding DNA (cDNA), or a DNA plasmid). In certain embodiments, the nucleic acid is an mRNA. In certain embodiments, the nucleic acid is a circular RNA. In certain embodiments, the nucleic acid is a DNA plasmid (pDNA).

In certain embodiments, the nucleic acid is a mixture of nucleic acids. For example, the nucleic acid may be a mixture of at least two (e.g., two, three, or four) nucleic acids. In certain embodiments, the nucleic acid is a mixture of at least two RNAs, such as a mixture of at least two miRNAs, and may be a mixture of two or three miRNAs that are different from one another. In certain embodiments, the nucleic acid is a mixture of three nucleic acids (e.g., miRNAs) that are different from one another.

The polymer nanoparticles described herein are capable of interacting with (e.g., encapsulating or complexing with) nucleic acids. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency is about 80% to about 95%.

In certain embodiments, the transfection efficiency is at least about 1%, or at least about 5%, or at least about 10% into cells, for example mammalian cells. In certain embodiments, the transfection efficiency is about 5% to about 35%, about 5% to about 30%, about 10% to about 30%, about 10% to about 25%, or about 15% to about 25%. In some embodiments, the transfection efficiency is at least about 30% or at least about 50%. In some embodiments, the transfection efficiency is about 30% to about 95%.

In some embodiments, the nucleic acid (e.g., each nucleic acid if a mixture of nucleic acids, for example miRNA) is from about 10 bp to about 200 bp. In some embodiments, the nucleic acid (e.g., RNA such as miRNA) is about 10 bp to about 100 bp, about 10 bp to about 50 bp, or about 10 bp to about 40 bp. In some embodiments, the nucleic acid (e.g., miRNA) is about 15 bp to about 30 bp.

In certain embodiments, the ratio of the nanoparticle to the nucleic acid (e.g., the total weight of nucleic acid if a mixture of nucleic acids) is about 5:1 to about 70:1 by weight, about 10:1 to about 70:1, or about 30:1 to about 70:1. For example, the ratio of nanoparticle to nucleic acid may be about 60:1 by weight. For example, the final mass of nanoparticle may be about 15 μg by weight with a mass for the total nucleic acid of about 0.25 μg (e.g., about 0.8 μg each if a mixture of three nucleic acids) or about 0.03 nmol.

In certain embodiments, the relative fold change of a target nucleic acid increases after transfection. For example, in some embodiments when the relative fold change for the expression of micro RNA miR-145 and miR-34a was found to be increased more than 100-fold for some nanoparticles while the fold change was reduced for the miRNA inhibitor, miR-21, when compared to controls (qPCR). For example, the relative fold change of the nucleotide could be about 1000 to about 2200, or about 2000 to about 7000. In another example, the relative fold change of the nucleotide is about 1 to about 10, or about 10 to about 40. In another example, the relative fold change of the miRNA is about 0 to about 1, or about 1 to about 2. For example, the relative fold change of the miRNA could be about 1000 to about 2200, or about 2000 to about 7000. In another example, the relative fold change of the miRNA is about 1 to about 10, or about 10 to about 40. In another example, the relative fold change of the miRNA is about 0 to about 1, or about 1 to about 2.

In some embodiments, the polymer nanoparticles and the complexed nucleic acid improve wound closure. For example, the wound closure in 36 hours could be by about 25% to about 85%, or about 35% to about 75%, or about 50% to about 100%.

In certain embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as lung cells. The compositions described herein can be used for treating disease, including lung diseases (e.g., lung cancer).

In some embodiments, a method of treating a patient with a disease is provided, comprising administering to the patient the polymer nanoparticle identified in the in vivo screening method, wherein the polymer nanoparticle further comprises a drug payload, such as a nucleic acid and treating the disease in the patient.

In various embodiments, any suitable route for administration of polymer nanoparticles associated with nucleic acid constructs for the method of in vivo screening for the polymer nanoparticle associated with a nucleic acid construct, or for the method of treatment can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In various embodiments, cell or tissue samples may be analyzed for the presence of the polymer nanoparticle associated with the nucleic acid constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal. Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In various embodiments, payloads may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

In some embodiments, the block copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via a high affinity, non-covalent bond, avidin-streptavidin conjugation. In some embodiments, the block copolymer can be associated with a DNA molecule, in particular a nucleic acid construct of the present disclosure, by direct covalent attachment through, for example, an amide bond.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C (═S) S—R) between chains and the propagating radicals. The overall process is comprised of the insertion of monomers between the R— and Z—C (═S) S-groups of a RAFT agent (CTA), which form the a and @ end-group of the majority of the resulting polymeric chains. Suitable CTAs for use in connection with the present disclosure include but are not limited to trithiocarbonates (Z═S-adlkyl), dithiobenzoates (Z═Ph), dithiocarbamate (Z═N-alkyl), xanthates (Z═O-alkyl), and the like. (See, Sébastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (˜pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis (carboxymethyl) trithiocarbonate, bis (2-amino-2-oxoethyl) trithiocarbonate, bis[4-(2-hydroxyethoxycarbonyl)benzyl] trithiocarbonate, 4-cyano-4-(ethylsulfanylthiocarbonyl) sulfanylvpentanoic acid, 4-cyano-4-((phenylcarbonothioyl)thio) pentanoic acid, and 4-cyano-4-[(dodecylsulfanylthiocarbonyl)sulfanyl] pentanoic acid, 4-cyano-4-(thiobenzoylthio) pentanoic acid, 2-cyano-2-propyl benzodithioate, cyanomethyl methyl (phenyl) carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In another illustrative embodiment, the polymer nanoparticle composition can be coated with one or more polymers to protect the compositions from immune responses or to enhance endosomal escape. In one embodiment, the one or more polymers used for coating comprise polyethylene glycol. In another embodiment, the one or more polymers used for coating comprise polyethylene glycol poly-L-lysine. In yet another embodiment, the one or more polymers used for coating comprise polyethylenimine. In an additional embodiment, the one or more polymers used for coating comprise polyethylene glycol poly-L-lysine and polyethylenimine.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), HexaneIsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

### Additional Embodiments

1. A block copolymer comprising

a first block comprising a homopolymer of monomer units represented by formula I:

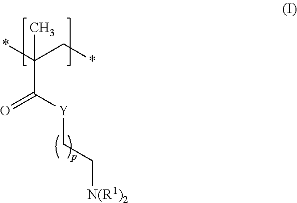

or a salt thereof, wherein:
p is 0 or an integer selected from 1-3,
each R¹ is individually selected from H or alkyl;
Y is O or NH; and
wherein the * represent the connecting points to the rest of the block copolymer; and

a second block comprising either:
(i) a homopolymer comprising monomer units represented by formula II:

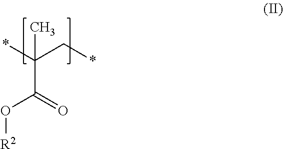

or a salt thereof, wherein:
R² is alkyl optionally substituted by hydroxy; and
wherein each * represents a connecting point to the rest of the block copolymer; or
(ii) a copolymer comprising at least two different monomer units, each represented by formula III:

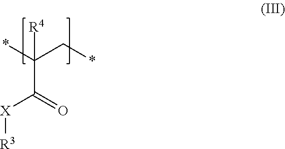

or a salt thereof, wherein:
X is O or NR⁵;
R³ is alkyl optionally substituted by amino;
R⁴ is H or alkyl; and
R⁵ is alkyl;
wherein each * represents a connecting point to the rest of the block copolymer.

2. The block copolymer of embodiment 1, wherein p is 1.

3. The block copolymer of embodiment 1 or 2, wherein each R¹ is C₁-C₃ alkyl such as methyl.

4. The block copolymer of embodiment 1 or 2, wherein each R¹ is H.

5. The block copolymer of any one of the preceding embodiments, wherein Y is O.

6. The block copolymer of any one of embodiments 1 to 4, wherein Y is NH.

7. The block copolymer of any one of the preceding embodiments, wherein the second block comprises a homopolymer.

8. The block copolymer of any one of the preceding embodiments, wherein R² is C₁-C₆ alkyl.

9. The block copolymer of any one of the preceding embodiments, wherein R² is butyl.

10. The block copolymer of any one of the preceding embodiments, wherein R² is ethyl.

11. The block copolymer of any one of the preceding embodiments, wherein R² is unsubstituted.

12. The block copolymer of any one of embodiments 1-10, wherein R² is substituted by hydroxy.

13. The block copolymer of any one of embodiments 1-6, wherein the second block comprises a copolymer.

14. The block copolymer of any one of embodiments 1-6 or 13, wherein R³ is C₁-C₆ alkyl.

15. The block copolymer of any one of embodiments 1-6, 13, or 14, wherein R³ is butyl.

16. The block copolymer of any one of embodiments 1-6, or 13, or 14, wherein R³ is ethyl.

17. The block copolymer of any one of embodiments 1-6 or 13-16, wherein R³ is unsubstituted.

18. The block copolymer of any one of the preceding embodiments, wherein X is O.

19. The block copolymer of any one of embodiments 1-17, wherein X is NR⁵.

20. The block copolymer of any one of the preceding embodiments, wherein R⁵ is C₁-C₆ alkyl.

21. The block copolymer of any one of the preceding embodiments, wherein R⁵ is ethyl.

22. The block copolymer of any one of the preceding embodiments, wherein R⁴ is H.

23. The block copolymer of any one of embodiments 1-21, wherein R⁴ is alkyl (e.g., methyl).

24. A block copolymer comprising a first block and a second block, wherein:
the first block comprises a homopolymer of poly (2-dimethylaminoethyl methacrylate) p(DMAEMA) or poly (2-aminoethyl methacrylamide) p(AEMA); and
the second block comprises either:
   (i) a homopolymer of poly (butylmethacrylate) p (BMA) or a homopolymer of poly (2-hydroxyethylmethacrylate) p(HEMA); or
   (ii) a copolymer comprising at least two monomer units selected from DMAEMA, BMA, and diethylacrylamide (DEAAM).

25. The block copolymer of embodiment 24, wherein the block copolymer is selected from:
(i) an AEMA homopolymer first block and a BMA homopolymer second block;
(ii) an AEMA homopolymer first block and a HEMA homopolymer second block; or
(iii) a DMAEMA homopolymer first block and a BMA/DEAAM copolymer second block.

26. The block copolymer of any one of the preceding embodiments, wherein the first block has a molecular weight of about 2 kDa to about 40 kDa.

27. The block copolymer of any one of the preceding embodiments, wherein the first block has a molecular weight of about 25 kDa to about 40 kDa.

28. The block copolymer of any one of the preceding embodiments, wherein the first block has a molecular weight of about 25 kDa to about 35 kDa.

29. The block copolymer of any one of embodiments 1 to 26, wherein the first block has a molecular weight of about 20 kDa to about 40 kDa.

30. The block copolymer of any one of embodiments 1 to 26, wherein the first block has a molecular weight of about 2 kDa to about 12 kDa.

31. The block copolymer of any one of the preceding embodiments, wherein the first block has a degree of polymerization (DP) of about 20 to about 250.

32. The block copolymer of any one of the preceding embodiments, wherein the first block has a degree of polymerization (DP) of about 150 to about 250.

33. The block copolymer of one of embodiments 1 to 30, wherein the first block has a degree of polymerization (DP) of about 20 to about 90.

34. The block copolymer of any one of the preceding embodiments, wherein the second block copolymer is a copolymer of at least two monomers, wherein the molar percent of the first monomer is about 30% to about 70% and the molar percent of the second monomer is about 70% to about 30%.

35. The block copolymer of any one of the preceding embodiments, wherein the second block copolymer is a copolymer of at least two monomers, wherein the molar percent of the first monomer is about 50% to about 60% and the molar percent of the second monomer is about 50% to about 40%.

36. A polymer nanoparticle comprising a block copolymer according to any one of the preceding embodiments.

37. The polymer nanoparticle according to embodiment 36, wherein the diameter (e.g., a hydrodynamic diameter as measured by DLS) of the block copolymer is about 200 nm to about 1000 nm, about 800 nm to about 1000 nm, or about 200 nm to about 800 nm.

38. The polymer nanoparticle according to embodiment 36 or 37, wherein the diameter of the block copolymer increases following lyophilization.

39. The polymer nanoparticle according to embodiment 36 or 37, wherein the diameter of the block copolymer does not change following lyophilization.

40. The polymer nanoparticle according to any one of embodiments 36-39, wherein the block copolymer has a measured molecular weight by NMR of about 2 to about 60 kDa.

41. The polymer nanoparticle according to any one of embodiments 36-39, wherein the block copolymer has a measured molecular weight by NMR of about 35 to about 45 kDa.

42. A composition comprising:
a polymer nanoparticle according to any one of embodiments 36-41, and
at least one nucleic acid complexed to the polymer nanoparticle.

43. The composition of embodiment 42, wherein the nucleic acid is a miRNA or an anti-miRNA.

44. The composition of embodiment 42 or 43, wherein the nucleic acid is a miRNA.

45. The composition of any one of embodiments 42-44, wherein the composition comprises at least two different nucleic acids (e.g., at least two different miRNAs).

46. The composition of any one of embodiments 43-45, wherein the miRNA comprises a sequence selected from the group consisting of SEQ ID NO: 1, SEQ ID NO: 2, and SEQ ID NO: 3, or a combination thereof.

47. The composition of any one of embodiments 43-45, wherein the composition comprises a first nucleic acid comprising SEQ ID NO: 1, a second nucleic acid comprising SEQ ID NO: 2, and a third nucleic acid comprising SEQ ID NO: 3

48. The composition of embodiment 42, comprising a miRNA mimic and a miRNA inhibitor.

49. The composition according to any one of embodiments 42-48, wherein the composition is solid, optionally wherein it has been lyophilized and further optionally including a cryoprotectant.

50. The composition according to any one of embodiments 42-49, wherein the nucleic acid complexed to the polymer nanoparticle transfects a cell.

51. The composition according to any one of embodiments 42-50, wherein the composition has a cell transfection efficiency of about 30% to about 95%.

52. The composition according to embodiment 49 or 50, wherein the cell is a mammalian cell.

53. The composition according to any one of embodiments 49-52, wherein the cell is a cancer cell (e.g., a lung cancer cell).

54. The composition of any one of embodiments 42-53, wherein the nucleic acid changes nucleotide expression (e.g., changes expression of target RNAs or miRNAs).

55. A method of treating a disease in a patient in need thereof, the method comprising administering a composition according to any one of embodiments 42-54.

56. The method of embodiment 55, wherein the polymer nanoparticles are delivered to the lungs of the patient.

57. The method of embodiment 55, wherein the disease is a lung disease.

### EXAMPLES

### Polymer Nanoparticle Synthesis

Diblock copolymers were synthesized similarly as described in U.S. Patent Application Publication No. 2022/0175812, the entirety of which is incorporated by reference herein, with some modifications using reversible addition-fragmentation chain transfer (RAFT) polymerization with reagents and amounts listed in Table 2. Block 1 reagents, including 2-(N,N-Dimethylamino)ethyl methacrylate (DMAEMA) as the monomer, 4-Cyano-4-[(ethylsulfanylthiocarbonyl)sulfanyl]pentanoic acid (ECT) as the chain transfer agent (CTA), 2,2′-Azobis(2-methylpropionitrile (AIBN) as the initiator, and N,N-Dimethylformamide (DMF) as the solvent, were combined in a 100 mL round-bottom flasks, purged with argon, and heated to 60° C. for 24 hours using either an oil bath. The reaction products were purified using four 80:20 pentane: ether precipitation washes and centrifugation cycles and dried in vacuo. The Block 1 products were characterized for molecular weight using GPC, and used as the macroRAFT agents for Block 2, and the calculated reagent volumes (as calculated based on theoretical molecular weight information for Block 1) were combined with AIBN initiator and various acrylate monomers in Corning 96-well polypropylene cluster tubes for the Block 2 reactions. The reaction mixtures were argon purged before being heated at 70° C. for 24 hours. The reaction products were purified using either four 80:20 pentane: ether precipitation washes and centrifugation cycles or three 80:20 pentane: diethyl ether washes and centrifugation cycles followed by drying in vacuo. Finally, the purified materials were lyophilized for 3-4 days and stored at room temperature for experimental use.

Block copolymers were prepared using Reversible Addition-Fragmentation Chain Transfer (RAFT) polymerization, using the monomers shown in Table 1 below. Block copolymers included:

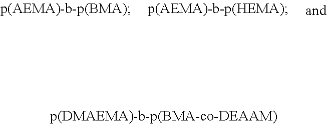

with block 1 degree of polymerization ranging from about 20 to about 230 (MW range from about 3 to about 40 kDa), and block 2 degree of polymerization ranging from about 2 to about 355 (MW range from about 0.2 to about 12 kDa).

TABLE 1
Monomers used in the nanoparticle forming polymers (PNPs).

| Monomer Name | Abbreviation | CAS | Structure |
|---|---|---|---|
| Butyl methacrylate | BMA | 97-88-1 | 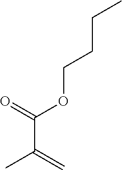 |
| Dimethylaminoethyl methacrylate | DMAEMA | 2867- 47-2 | 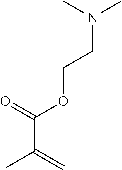 |
| 2-Aminoethyl methacrylamide | AEMA | 76259- 32-0 | 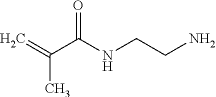 |
| N,N-Diethylacrylamide | DEAAM | 2675- 94-7 | 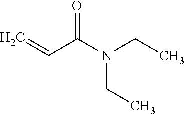 |
| Hydroxyethylmethacrylate | HEMA | 868-77- 9 | 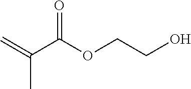 |

TABLE 2
miRNA PNP compositions.

| | Block 1 | | | | | | | | | | | | |
|---|---|---|---|---|---|---|---|---|---|---|---|---|---|
| | Composition | | | | | Diblock | | | | | | | |
| | | B1 | Block 2 Composition | | | Composition | | B2 | | | | | NMR |
| | | Mol. | | | Mol | | Mol | Mol. | | Mol | Mol | Mol | measured |
| ID | B1 | Wt. | DP B1 | | % | | % | Wt. | DP B2 | % | % | % | Mol. Wt. |
| ID | Monomer | (kDa) | Overall | B2M1 | B2M1 | B2M2 | B2M2 | (kDa) | Overall | B1 | B2M1 | B2M2 | (kDa) |
| 1 | DMAEMA | 25.93 | 165 | BMA | 56 | DEAAM | 44 | 10.53 | 78 | 68 | 18 | 14 | 36.46 |
| 2 | DMAEMA | 36.05 | 229 | BMA | 55 | DEAAM | 45 | 8.75 | 65 | 78 | 12 | 10 | 44.80 |
| 3 | AEMA | 8.06 | 62 | BMA | 100 | N/A | 0 | 0.47 | 3 | 95 | 5 | 0 | 8.53 |
| 4 | AEMA | 11.37 | 88 | BMA | 100 | N/A | 0 | 0.66 | 5 | 95 | 5 | 0 | 12.03 |
| 5 | AEMA | 3.46 | 27 | BMA | 100 | N/A | 0 | 0.29 | 2 | 93 | 7 | 0 | 3.75 |
| 6 | AEMA | 8.06 | 62 | HEMA | 100 | N/A | 0 | 4.57 | 35 | 64 | 36 | 0 | 12.63 |
| 7 | AEMA | 11.37 | 88 | HEMA | 100 | N/A | 0 | 45.83 | 352 | 20 | 80 | 0 | 57.20 |
| 8 | AEMA | 3.46 | 27 | HEMA | 100 | N/A | 0 | 0.48 | 4 | 88 | 12 | 0 | 3.94 |

### Dynamic Light Scattering

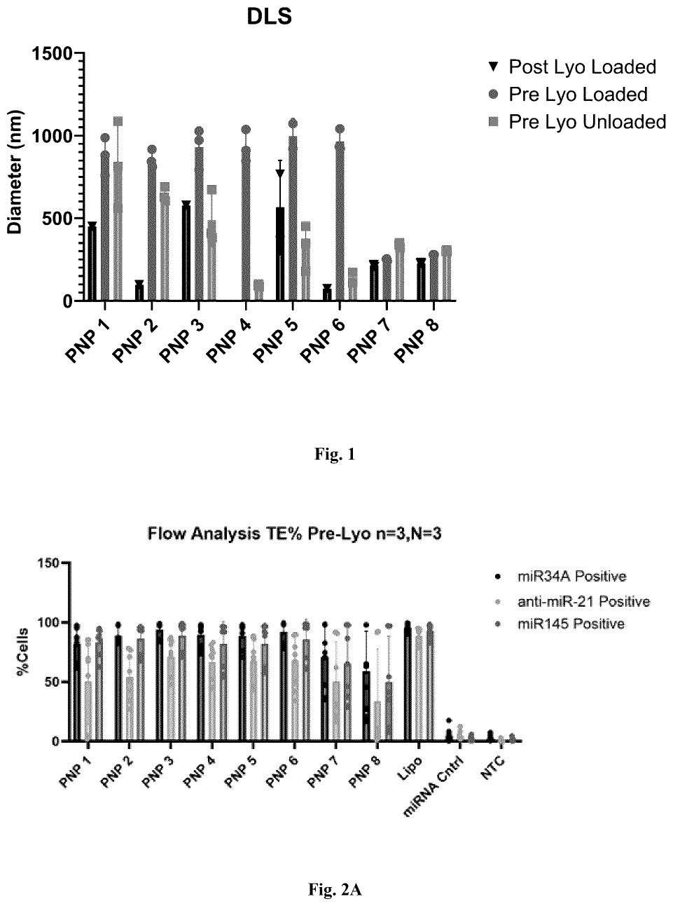

The particle size of the polymers, with and without miRNA or anti-miRNA payloads complexed, was measured via dynamic light scattering using a Wyatt DynaPro III. Results are shown in FIG. 1.

### Nucleic Acid Loading

In one example, miRNA was complexed at 0.13 umol/L with the PNPs by mixing the PNPs at a final concentration of 0.05 mg/mL and allowing the PNPs and miRNA to form an electrostatic complex at room temperature

TABLE 3
PNP IDs for miRNA analysis

| miRNA Cocktail Cargo | | |
|---|---|---|
| miRNA | Sequence | SEQ ID NO |
| miRNA | rG*rU*rC* rCrArG rUrUrU | SEQ ID |
| 145 | rUrCrC rCrArG rGrArA | NO.: 1 |
| | rUrC*rC* rC*rU/3Cy5Sp/ | |
| miRNA | rC*rA*A* rUrCrA rGrCrA | SEQ ID |
| 34a | rArGrU rArUrA rCrUrG | NO.: 2 |
| | rC*rC*rC* rU/3ATTO488N/ | |
| Anti- | rU*rC*rA rArCrA rUrCrA | SEQ ID |
| miRNA 21 | rGrUrC rUrGr rUrArA | NO.: 3 |
| | rG*rC*rU* rA/3TYE563/ | |

*=phosphorothioate modification

### Transfection Efficiency in A549 Cells

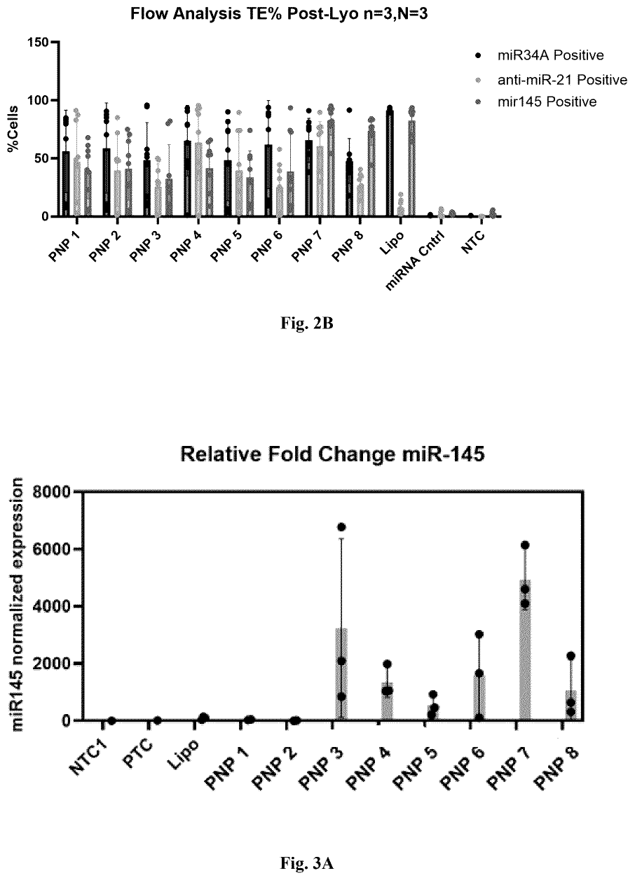

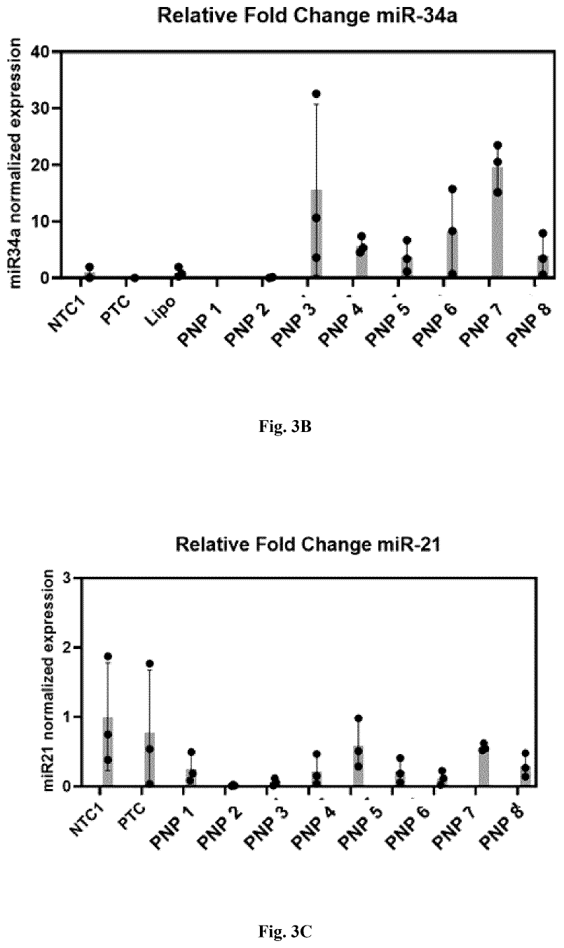

To measure the in vitro transfection efficiency, the PNPs were loaded with a cocktail of each miRNA according to Table 3. A549 lung cancer cells were cultured and plated on 48 well plates to analyze the efficiency of PNP delivery. PNPs were loaded with a miRNA cocktail of miRNA-34a, miRNA-145 and AMO-21 at a final concentration of 0.13 μmol/L. Cells were dosed with each loaded PNP in triplicate 24 hours after plating at a PNP concentration of 0.05 mg/mL. The six PNPs were also loaded with a non-functional scrambled miRNA control to assess any PNP-specific effects. The cells were processed and analyzed 48 hrs post dose via flow cytometry (FIG. 2.) and RT-qPCR (FIG. 3A-C.)

### Wound Closure Delivery

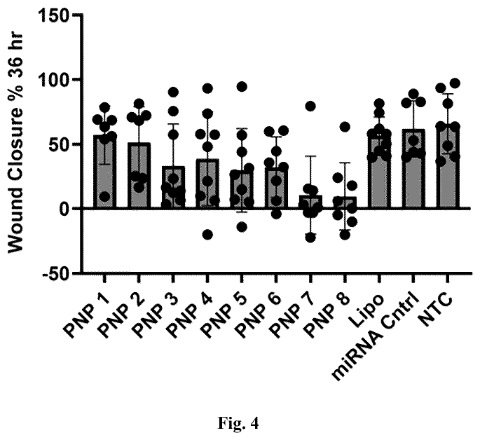

A549 cells were plated in 48 well plates. A scratch was induced post-transfection into each well and cell migration monitored over time via imaging. The images were analyzed via imageJ to determine the area of the wound after 36 hours. Results are shown in FIG. 4.

1. A block copolymer comprising a first block and a second block, wherein:
the first block comprises a homopolymer of poly (2-dimethylaminoethyl methacrylate) p(DMAEMA) or poly (2-aminoethyl methacrylamide) p(AEMA); and
the second block comprises either:
(i) a homopolymer of poly (butylmethacrylate) p (BMA) or a homopolymer of poly (2-hydroxyethylmethacrylate) p(HEMA); or
(ii) a copolymer comprising at least two monomer units selected from DMAEMA, BMA, and diethylacrylamide (DEAAM).

2. The block copolymer of claim 1, wherein the block copolymer comprises:
(i) an AEMA homopolymer first block and a BMA homopolymer second block;
(ii) an AEMA homopolymer first block and a HEMA homopolymer second block; or
(iii) a DMAEMA homopolymer first block and a BMA/DEAAM copolymer second block.

3. The block copolymer of claim 1, wherein the block copolymer comprises an AEMA homopolymer first block and a BMA homopolymer second block.

4. The block copolymer of claim 1, wherein the block copolymer comprises an AEMA homopolymer first block and a HEMA homopolymer second block.

5. The block copolymer of claim 1, wherein the block copolymer comprises a DMAEMA homopolymer first block and a BMA/DEAAM copolymer second block.

6. The block copolymer of claim 1, wherein the first block has a molecular weight of about 2 kDa to about 40 kDa.

7. The block copolymer of claim 1, wherein the first block has a molecular weight of about 25 kDa to about 40 kDa.

8. The block copolymer of claim 1, wherein the first block has a molecular weight of about 2 kDa to about 12 kDa.

9. The block copolymer of claim 1, wherein the first block has a degree of polymerization (DP) of about 20 to about 250.

10. The block copolymer of claim 1, wherein the first block has a degree of polymerization (DP) of about 150 to about 250.

11. The block copolymer of claim 1, wherein the second block copolymer and the molar percent of the first monomer is about 30% to about 70% and the molar percent of the second monomer is about 70% to about 30%.

12. A polymer nanoparticle comprising the block copolymer of claim 1.

13. The polymer nanoparticle of claim 12, wherein the diameter of the nanoparticle is about 200 nm to about 1000 nm.

14. A composition comprising:
the polymer nanoparticle of claim 12, and
at least one nucleic acid complexed to the polymer nanoparticle.

15. The composition of claim 14, wherein the nucleic acid is a miRNA.

16. The composition of claim 14, wherein the composition comprises at least two different miRNAs.

17. The composition of claim 14, wherein the at least one nucleic acid comprises a sequence selected from the group consisting of SEQ ID NO: 1, SEQ ID NO: 2, and SEQ ID NO: 3.

18. The composition of claim 14, wherein the composition comprises a first nucleic acid comprising SEQ ID NO: 1, a second nucleic acid comprising SEQ ID NO: 2, and a third nucleic acid comprising SEQ ID NO: 3.

19. A method of treating a disease in a patient in need thereof, the method comprising administering the composition of claim 14 to the patient.

20. The method of claim 19, wherein the polymer nanoparticles are delivered to the lungs of the patient.
